(19) United States
(12) Patent Application Publication
(10) Pub. No.: US 2025/0041191 A1
(43) Pub. Date: Feb. 6, 2025

(54) COMPOSITIONS FOR EVENING SKIN TONE AND USES THEREOF

(71) Applicant: RoC OPCO LLC, New York, NY (US)

(72) Inventors: Arthur Pellegrino, Newtown, PA (US); Michelle Taylor, Pacific Palisades, CA (US)

(21) Appl. No.: 18/793,411

(22) Filed: Aug. 2, 2024

Related U.S. Application Data
(60) Provisional application No. 63/530,436, filed on Aug. 2, 2023.

(51) Int. Cl. A61K 8/49; A61K 8/34; A61K 8/44; A61K 8/67; A61K 8/9789; A61Q 19/02

(52) U.S. Cl. CPC A61K 8/498; A61K 8/347; A61K 8/44; A61K 8/675; A61K 8/9789; A61Q 19/02

(57) ABSTRACT

Disclosed herein are compositions for evening skin tone comprising at least one tyrosine formation inhibitor, at least one tyrosinase inhibitor, and at least one agent that reduces melanosome transfer from melanocytes to keratinocytes, wherein the composition does not comprise hydroquinone. Disclosed uses of the compositions include methods for persistently evening skin tone.

This application claims priority to U.S. Provisional Application No. 63/530,436, filed Aug. 2, 2023, the entire contents of which are incorporated herein by reference.

Disclosed herein are compositions for evening skin tone including at least one tyrosine formation inhibitor, at least one tyrosinase inhibitor, and at least one agent that reduces melanosome transfer from melanocytes to keratinocytes, wherein the composition does not include hydroquinone. Disclosed uses of the compositions include methods of evening skin tone.

Skin tone evenness relates to the uniformity of skin pigmentation. Many factors may affect skin tone evenness such as sun exposure and aging. It may be desirable to increase skin tone evenness by, e.g., reducing the number, size, and/or intensity of dark spots.

Compositions for evening skin tone commonly use hydroquinone because of its effectiveness in evening skin tone. However, the use of hydroquinone can sometimes cause undesirable side-effects such as, for example, rashes, swelling, and skin discoloration. Thus, there is a need for skin evening compositions that reduce or avoid the use of hydroquinone while remaining at least as effective. Accordingly, there is a need for improved compositions for evening skin tone.

### SUMMARY

Disclosed are compositions for evening skin tone including, wherein the compositions include at least one tyrosine formation inhibitor, at least one tyrosinase inhibitor, and at least one agent that reduces melanosome transfer from melanocytes to keratinocytes, and wherein the compositions do not include hydroquinone.

In some embodiments, the composition includes the at least one tyrosine formation inhibitor, the at least one tyrosinase inhibitor, and the at least one agent that reduces melanosome transfer from melanocytes to keratinocytes in an amount sufficient to reduce the number, size, and/or intensity of dark spots.

In some embodiments, the at least one tyrosine formation inhibitor includes kojic acid or an ester thereof, the at least one tyrosinase inhibitor includes tranexamic acid, an ester of tranexamic acid, licorice root extract, hexyl resorcinol, or a combination thereof, and the at least one agent that reduces melanosome transfer from melanocytes to keratinocytes includes niacinamide.

In some embodiments, the composition includes kojic acid or an ester of kojic acid, tranexamic acid or an ester of tranexamic acid, licorice root extract, hexyl resorcinol, and niacinamide.

In some embodiments, the composition includes from 0.2 wt. % to 2.0 wt. % kojic dipalmitate, from 1.0 wt. % to 10.0 wt. % tranexamic acid, from 0.001 wt. % to 0.05 wt. % licorice root extract, from 0.10 wt. % to 1.0 wt. % hexyl resorcinol, and from 1.0 wt. % to 15.0 wt. % niacinamide.

In some embodiments, the composition includes about 1.0 wt. % kojic dipalmitate, about 5.0 wt. % tranexamic acid, about 0.01 wt. % licorice root extract, about 0.40 wt. % hexyl resorcinol, and about 5.0 wt. % niacinamide.

Also disclosed are methods of evening skin tone including applying a disclosed composition the skin.

In some embodiments, the composition is applied to the skin in an amount sufficient to reduce the number, size, and/or intensity of dark spots.

In some embodiments, the reduction of dark spots is more effective than treatment with a hydroquinone-containing composition.

### Definitions

As used herein, the term “skin tone evenness” refers to the uniformity of pigmentation of the skin.

As used herein, a “tyrosine formation inhibitor” is any compound that reduces tyrosine biosynthesis in a subject.

As used herein, a “tyrosinase inhibitor” is any compound that reduces the activity of a tyrosinase enzyme.

While any one compound may be a tyrosine formation inhibitor, a tyrosinase inhibitor, and/or an agent that reduces melanosome transfer from melanocytes to keratinocytes, as used herein, a “composition comprising at least one tyrosine formation inhibitor, at least one tyrosinase inhibitor, and at least one agent that reduces melanosome transfer from melanocytes to keratinocytes” has at least three different compounds wherein a first one of the compounds is at least a tyrosine formation inhibitor, a second one of the compounds is at least a tyrosine formation inhibitor, and a third one of the compounds is at least an agent that reduces melanosome transfer from melanocytes to keratinocytes.

As used herein, the “intensity” of a dark spot refers to the average darkness of the dark spot compared to that of the surrounding skin.

Unless otherwise stated, all parts and percentages are by weight. As used herein, the term “about” refers to plus or minus 10% of the indicated value. Unless otherwise stated, weight percentages are provided based on the total amount of the composition in which they are described. As used herein, the singular forms “a,” “an,” and “the” include plural referents unless stated otherwise.

### DETAILED DESCRIPTION

### Compositions

Compositions of the present disclosure may be useful for evening skin tone. Ingredients used for evening skin tone may serve one or more functions such as inhibiting tyrosine formation, inhibiting tyrosinase, and reducing melanosome transfer from melanocytes to keratinocytes. Such compositions include at least one tyrosine formation inhibitor, at least one tyrosinase inhibitor, and at least one agent that reduces melanosome transfer from melanocytes to keratinocytes.

Without wishing to be bound by theory, while some compounds may or may not be known in isolation to improve skin tone evenness, it is believed that the combination of at least one tyrosine formation inhibitor, at least one tyrosinase inhibitor, and at least one agent that reduces melanosome transfer from melanocytes to keratinocytes having, e.g., different mechanisms of action provides enhanced performance in reducing the number, size, and/or intensity of dark spots as compared to any single subcomponent of the composition insolation.

In some embodiments, the at least one tyrosine formation inhibitor includes kojic acid or an ester thereof, the at least one tyrosinase inhibitor includes tranexamic acid, an ester of tranexamic acid, licorice root extract, hexyl resorcinol, or a combination thereof, and the at least one agent that reduces melanosome transfer from melanocytes to keratinocytes includes niacinamide.

The disclosed compositions may include the at least one tyrosine formation inhibitor, the at least one tyrosinase inhibitor, and the at least one agent that reduces melanosome transfer from melanocytes in various amounts. For example, in some embodiments, the composition includes the at least one tyrosine formation inhibitor, the at least one tyrosinase inhibitor, and the at least one agent that reduces melanosome transfer from melanocytes to keratinocytes in amounts sufficient to reduce the number, size, and/or intensity of dark spots.

In some embodiments, the composition includes a tyrosine formation inhibitor in an amount ranging from 0.1 wt. % to 5.0 wt. % by total weight of the composition. In some embodiments, the composition includes a tyrosine formation inhibitor in an amount ranging from 0.2 wt. % to 2.0 wt. % by total weight of the composition. In some embodiments, the composition includes a tyrosinase inhibitor in an amount ranging from 0.001 wt. % to 15.0 wt. % by total weight of the composition. In some embodiments, the composition includes an agent that reduces melanosome transfer from melanocytes to keratinocytes in an amount ranging from 1.0 wt. % to 15.0 wt. % by total weight of the composition.

In some embodiments, the composition includes a total amount of tyrosine formation inhibitor ranging from 0.1 wt. % to 5.0 wt. % by total weight of the composition, a total amount of tyrosinase inhibitor ranging from 0.001 wt. % to 15.0 wt. % by total weight of the composition, and a total amount of agent that reduces melanosome transfer from melanocytes to keratinocytes ranging from 1.0 wt. % to 15.0 wt. % by total weight of the composition.

In some embodiments, the composition includes at least one tyrosine formation inhibitor in an amount ranging from 0.2 wt. % to 2.0 wt. % by total weight of the composition, at least one tyrosinase inhibitor in an amount ranging from 0.001 wt. % to 15.0 wt. % by total weight of the composition, and at least one agent that reduces melanosome transfer from melanocytes to keratinocytes in an amount ranging from 1.0 wt. % to 15.0 wt. % by total weight of the composition.

In some embodiments, the composition includes: from 0.2 wt. % to 2.0 wt. % kojic dipalmitate, from 1.0 wt. % to 10.0 wt. % tranexamic acid, from 0.001 wt. % to 0.05 wt. % licorice root extract, from 0.10 wt. % to 1.0 wt. % hexyl resorcinol, and from 2.5 wt. % to 7.5 wt. % niacinamide. For example, in some embodiments, the composition includes about 1.0 wt. % kojic dipalmitate, about 5.0 wt. % tranexamic acid, about 0.01 wt. % licorice root extract, about 0.40 wt. % hexyl resorcinol, and about 5.0 wt. % niacinamide.

In some embodiments, the composition includes from 0.2 wt. % to 2.0 wt. % kojic dipalmitate. In some embodiments, the composition includes from 1.0 wt. % to 10.0 wt. % tranexamic acid. In some embodiments, the composition includes from 0.001 wt. % to 0.05 wt. % licorice root extract. In some embodiments, the composition includes from 0.10 wt. % to 1.0 wt. % hexyl resorcinol. In some embodiments, the composition includes from 2.5 wt. % to 7.5 wt. % niacinamide.

In some embodiments, the composition includes about 1.0 wt. % kojic dipalmitate. In some embodiments, the composition includes about 5.0 wt. % tranexamic acid. In some embodiments, the composition includes about 0.01 wt. % licorice root extract. In some embodiments, the composition includes about 0.40 wt. % hexyl resorcinol. In some embodiments, the composition includes about 5.0 wt. % niacinamide.

In some embodiments, the composition includes about 1.0 wt. % kojic dipalmitate. In some embodiments, the composition includes about 3.0 wt. % cetyl tranexamate mesylate. In some embodiments, the composition includes about 0.01 wt. % licorice root extract. In some embodiments, the composition includes about 0.40 wt. % hexyl resorcinol. In some embodiments, the composition includes about 6.35 wt. % niacinamide.

In some embodiments, the composition includes a retinoid such as retinol. Retinol, other retinoids, and related compounds, e.g., Bakuchiol, have the added advantage of increasing cell turnover. In some embodiments, the retinol, other retinoid, or related compound is present in an amount of 0.001 wt. % to 1 wt. % of the formulation. In some embodiments, the composition does not include a retinoid such as retinol. In some embodiments, the composition does not include mandelic acid or an ester thereof. In some embodiments, the composition does not include azelaic acid or an ester thereof.

In some embodiments, the composition includes one or more exfoliants. In some embodiments, the one or more exfoliates are chosen from chemical exfoliants, enzyme exfoliants, and combinations thereof. In some embodiments, the chemical exfoliants are chosen from mandelic acid, glycolic acid, lactic acid, malic acid, tartaric acid, citric acid, salicylic acid, lactobionic acid, gluconolactone, galactose, HEPES (sulfonic acid) and combinations thereof. In some embodiments, the chemical exfoliant includes mandelic acid. In some embodiments, the enzyme exfoliants are chosen from a trypsin, a collagenase, and combinations thereof.

### Tyrosine Formation Inhibitors

Tyrosine formation inhibitors reduce tyrosine biosynthesis in a subject. In some embodiments, the composition includes at least one tyrosine formation inhibitor chosen from kojic acid, an ester of kojic acid, and combinations thereof. In some embodiments, the ester of kojic acid is kojic dipalmitate.

### Tyrosinase Inhibitors

Tyrosinase inhibitors reduce the activity of a tyrosinase enzyme. In some embodiments, the composition includes at least one tyrosinase inhibitor chosen from arbutin, tetrahydropyranyloxy phenol, azelaic acid, an ester of azelaic acid, a resorcinol (e.g., hexylresorcinol), ascorbic acid and derivatives thereof (including ascorbic acid esters such as ethyl vitamin C; and natural sources of vitamin C such as Terminalia Ferdinandiana fruit (kakadu plum) extract), ellagic acid, an ester of ellagic acid, mandelic acid, an ester of mandelic acid, tranexamic acid, an ester of tranexamic acid (e.g., cetyl tranexamate mesylate, licorice extract, a retinoid (e.g., retinol), bakuchiol, cinnamic acid hydroxypyridinone derivatives, and combinations thereof. In some embodiments, the tyrosinase inhibitor includes tranexamic acid, an ester of tranexamic acid, licorice root extract, hexyl resorcinol, or a combination thereof.

Agents that Reduce Melanosome Transfer from Melanocytes to Keratinocytes

Agents that reduce melanosome transfer from melanocytes to keratinocytes include agents that decrease the number of melanocytes. These include, for example, niacinamide, malassezin, and combinations thereof.

### Formulations of the Composition

In some embodiments, the composition is a cosmetic composition. The disclosed compositions may be formulated as, e.g., a topical ointment, a cream, a lotion, a serum, a moisturizer, a sunscreen, a cleanser, and/or a peel. Such compositions may, in addition to evening skin tone, improve, e.g., skin hydration, texture, firmness, and/or elasticity.

Compositions of the present disclosure may be formulated with one or more solvents and dermatologically acceptable excipients.

In some embodiments, the composition includes one or more excipients. Non-limiting examples of excipients include:
Emulsifiers (such as nonionic, cationic, anionic, or polymeric emulsifiers), surfactants (such as non-ionic, cationic, and anionic surfactants) and rheology modifiers, e.g., polyacrylate-13, polyisobutene, sorbitan isooctadecanoate, decyl glucoside, polysorbate 20, polyvinyl alcohol;
Delivery Agents, e.g. Lactic Acid/Glycolic Acid Copolymer (PLGA);
Humectants, emollients and other skin conditioning agents, e.g., squalene, dimethicone butylene glycol, caprylyl glycol, hexylene glycol, hydrolyzed vegetable protein, trehalose, trehalose, coconut alkanes, coco-caprylate/caprate, 1,2-hexanediol, ethylhexylglycerin;
pH modifiers and pH buffers, e.g., citric acid;
Preservatives and antimicrobial agents, e.g., phenoxyethanol, denatonium benzoate
Fragrance (such as fruit or plant extracts, for example, in the form of fragrances or essential oils) and D&C colors;
Antioxidants;
Antiaging agents including anti-wrinkle agents; and
Film-forming agents e.g., polysilicone-11, polymethylsilsesquioxane.

In some embodiments, the composition includes one or more solvents. For example, the composition may include one or more solvent chosen from water, water soluble solvents, water immiscible solvents, and combinations thereof. Water and water soluble solvents include, for example alcohols such as ethanol propanol, isopropanol, glycerin, and mixtures thereof. Water immiscible solvents include oils and waxes. As used herein, an oil is a water insoluble solvent such as mineral oil, plant oils (e.g., Helianthus Annuus (sunflower) seed oil, Carthamus Tinctorius (safflower) seed oil) and silicone oils, such as dimethicone and cyclomethicone. In some embodiments, the composition includes water and/or water-soluble solvents, and oils and/or water immiscible solvents.

In some embodiments, the composition is an emulsion, such as an oil-in-water emulsion or water-in-oil emulsion. The oil in the emulsion may be a carbon or hydrocarbon-based oil or a silicone-based oil, e.g., a silicone emulsion.

In some embodiments, the compositions are a solution, for example an aqueous solution, or a suspension in water or oil. An exemplary oil-in-water emulsion contains about 60 wt. % to 90 wt. % purified water and water soluble components and about 10 wt. % to about 40 wt. % components forming a water immiscible or oil phase. “Purified water” is water that does not contain ingredients which would be harmful to, or would cause adverse reactions to, the skin of a subject, such as a human. In some embodiments, distilled water and/or deionized water is used.

In some embodiments, the composition is a serum or cream. Serum, as used herein, refers to a product that is absorbed and penetrates into deeper layers of the skin. Serums may have a light low viscosity, non-greasy finish and high concentrations of active ingredients. In some embodiments, the composition includes at least about 60% water by weight. In some embodiments, the composition is an oil-in-water emulsion.

### Methods for Persistently Evening Skin Tone

Compositions of the disclosure may be useful in methods of evening skin tone. Such methods include, for example, applying a disclosed composition to the skin. The composition may be applied to different skin locations including, for example, on the arms, face, back, legs, and/or torso. Application of the compositions may be performed using various amounts of the composition. The composition may be applied at various frequencies including, for example, twice daily, daily, once every two to three days, or weekly. The composition may be applied at any given frequency for any duration such as, for example, at least one week, at least one month, at least one year.

As used herein, a “method for persistently evening skin tone” is a method for increasing skin tone uniformity by application of a composition to skin wherein an increased skin tone uniformity persists after the composition is removed from the skin. Compositions of the disclosure even skin tone (e.g., by reducing the number, size, and/or intensity of dark spots) and provide a persistent effect after one or more applications of the composition. Persistent evening of skin tone (e.g., by reducing the number, size, and/or intensity of dark spots) may result from a single application, or may be achieved after multiple applications over an extended period. Multiple applications may take place with a frequency and duration indicated herein. Although evening of skin tone (e.g., by reducing the number, size, and/or intensity of dark spots) persists after removal of the composition from the skin, unevenness (e.g., dark spots) may return over time if application of the composition is ceased.

According to a method of the present disclosure the composition is applied at a frequency and duration sufficient for an even skin tone to persistent for at least 30 minutes, at least 1 hour, at least 2 hours, at least 4 hours, at least 12 hours, or at least 1 day after removing the composition from the skin. The composition is applied at a frequency and duration sufficient for an even skin tone to persistent for at least 1, at least 2 days, at least 1 week, at least 2 weeks, or at least one month after application of the composition is ceased. According to embodiments The composition is applied to the skin once or twice each day for at least 1 day, at least 2 days, at least 1 week, at least 2 weeks, or at least 1 month.

In some embodiments, the composition is applied to the skin at an amount, frequency, and duration sufficient to reduce the number, size, and/or intensity of dark spots. For example, in some embodiments, the composition is applied daily for at least one week in an amount sufficient to reduce the number, size, and/or intensity of dark spots.

In some embodiments, the method is more effective in reducing dark spots than treatment with a hydroquinone-containing composition. For example, in some embodiments, the method is at least as effective in reducing dark spots than treatment with a hydroquinone-containing composition, wherein the hydroquinone-containing composition does not include one or more of kojic acid or an ester of kojic acid, tranexamic acid or an ester of tranexamic acid, licorice root extract, hexyl resorcinol, and niacinamide; wherein the hydroquinone-containing composition does not include two or more of kojic acid or an ester of kojic acid, tranexamic acid or an ester of tranexamic acid, licorice root extract, hexyl resorcinol, and niacinamide; or wherein the hydroquinone-containing composition does not include kojic acid or an ester of kojic acid, tranexamic acid or an ester of tranexamic acid, licorice root extract, hexyl resorcinol, and niacinamide. In some embodiments, the method is more effective in reducing dark spots than treatment with a hydroquinone-containing composition, wherein the hydroquinone-containing composition does not include one or more of kojic acid or an ester of kojic acid, tranexamic acid or an ester of tranexamic acid, licorice root extract, hexyl resorcinol, and niacinamide; wherein the hydroquinone-containing composition does not include two or more of kojic acid or an ester of kojic acid, tranexamic acid or an ester of tranexamic acid, licorice root extract, hexyl resorcinol, and niacinamide; or wherein the hydroquinone-containing composition does not include kojic acid or an ester of kojic acid, tranexamic acid or an ester of tranexamic acid, licorice root extract, hexyl resorcinol, and niacinamide.

### EXAMPLES

The following examples are provided for illustrative purposes only and are not intended to limit the scope of the disclosure.

### Example 1

Table 1 provides an exemplary formulation amounts of active ingredients that may be used in compositions according to the present disclosure. Wt % is based on the total weight of the composition.

TABLE 1

| Component | Exemplary | Exemplary | Exemplary |
|---|---|---|---|
| Function | Component | Range (wt. %) | Amount (wt. %) |
| tyrosine | kojic acid | 0.2-5.0 | 1.0 |
| formation | or ester, e.g. | | |
| inhibitor | kojic dipalmitate | | |
| tyrosinase | tranexamic | TOTAL- 1.0-12.0 | Total 5.0-5.5 |
| inhibitor | acid or ester | 1.0-10.0 | 5.0 |
| | licorice root extract | 0.001-0.05 | 0.01 |
| | hexyl resorcinol | 0.10-1.0 | 0.40 |
| agent that | niacinamide | 2.5-7.5 | 4.0-6.5 |
| reduces | | | |
| melanosome | | | |
| transfer from | | | |
| melanocytes to | | | |
| keratinocytes | | | |

### Example 2

Table 2 is an exemplary scrum formulation.

TABLE 2

| Component | % in formula |
|---|---|
| Water | q.s. |
| Tranexamic Acid | 5.00 |
| Dimethicone | 3.83 |
| Polysilicone-11 | 0.50 |
| Butylene Glycol | 5.300 |
| Decyl Glucoside | 0.01 |
| Caprylyl Glycol | 0.003 |
| Hexylene Glycol | 0.003 |
| Niacinamide | 5.00 |
| Hydrolyzed Vegetable Protein | 1.23 |
| Trehalose | 0.69 |
| Glycerin | 0.69 |
| Alcohol Denat. | 2.77 |
| T-Butyl Alcohol | 0.0036 |
| Denatonium Benzoate | 0.000018 |
| Coconut Alkanes | 1.88 |
| Coco-Caprylate/Caprate | 0.12 |
| Polyacrylate-13 | 0.75 to 1.05 |
| Polyisobutene | 0.33 to 0.48 |
| Polysorbate 20 | 0.03 to 0.105 |
| Sorbitan isooctadecanoate | 0.045 to 0.075 |
| Polymethylsilsesquioxane | 1.00 |
| Diamond Powder | 0.00 to 0.00100000 |
| Kojic Dipalmitate | 1.00 |
| Helianthus Annuus (Sunflower) Seed Oil | 0.54 |
| Carthamus Tinctorius (Safflower) Seed | 0.16 |
| Oil | |
| Morus Alba Leaf Extract | 0.16 |
| Squalane | 0.14 |
| Phenoxyethanol | 0.90 |
| Ethylhexylglycerin | 0.10 |
| 1,2-Hexanediol | 0.50 |
| Hexylresorcinol | 0.40 |
| Polysorbate 20 | 0.31 |
| Tetrahydrodiferuloylmethane | 0.25 |
| Citric Acid | 0.11 |
| Glycyrrhiza Glabra (Licorice) Root | 0.01 |
| Extract | |
| Lactic Acid/Glycolic Acid Copolymer | 0.0003 to 0.0005 |
| Palmitoyl sh-Octapeptide-24 Amide | 0.00024 to 0.00036 |
| Polyvinyl Alcohol | 0.00016 to 0.00024 |
| Palmitoyl sh-Tripeptide-5 Norisoleucyl | 0.00008 to 0.00012 |
| sh-Nonapeptide-1 | |

### Example 3

Table 3 is an exemplary scrum formulation.

TABLE 3

| Component | % in formula |
|---|---|
| Water | q.s. |
| Tranexamic Acid | 5.00 |
| Dimethicone | 3.83 |
| Polysilicone-11 | 0.50 |
| Butylene Glycol | 0.30 |
| Decyl Glucoside | 0.010 |
| Caprylyl Glycol | 0.0028 |
| Phenoxyethanol | 0.0028 |
| Hexylene Glycol | 0.0028 |
| Niacinamide | 5.00 |
| Butylene Glycol | 5.00 |
| Hydrolyzed Vegetable Protein | 1.23 |
| Trehalose | 0.69 |
| Glycerin | 0.69 |
| Alcohol Denat. | 2.77 |
| T-Butyl Alcohol | 0.0036 |
| Denatonium Benzoate | 0.000018 |
| Coconut Alkanes | 1.88 |
| Coco-Caprylate/Caprate | 0.12 |
| Polyacrylate-13 | 0.750 to 1.050 |
| Polyisobutene | 0.330 to 0.480 |
| Polysorbate 20 | 0.030 to 0.105 |
| Sorbitan isooctadecanoate | 0.045 to 0.075 |
| Kojic Dipalmitate | 1.00 |
| Helianthus Annuus (Sunflower) Seed Oil | 0.54 |
| Carthamus Tinctorius (Safflower) Seed | 0.16 |
| Oil | |
| Morus Alba Leaf Extract | 0.16 |
| Squalane | 0.14 |
| Phenoxyethanol | 0.90 |
| Ethylhexylglycerin | 0.10 |
| 1,2-Hexanediol | 0.50 |
| Hexylresorcinol | 0.40 |
| Polysorbate 20 | 0.31 |
| Tetrahydrodiferuloylmethane | 0.25 |
| Citric Acid | 0.11 |
| Glycyrrhiza Glabra (Licorice) Root | 0.01 |
| Extract | |
| Lactic Acid/Glycolic Acid Copolymer | 0.0003 to 0.0005 |
| Palmitoyl sh-Octapeptide-24 Amide | 0.00024 to 0.00036 |
| Polyvinyl Alcohol | 0.00016 to 0.00024 |
| Palmitoyl sh-Tripeptide-5 Norisoleucyl | 0.000080 to 0.00012 |
| sh-Nonapeptide-1 | |

### Example 4

Table 4 is an exemplary scrum formulation.

| Component | Wt. % in formula |
|---|---|
| Water (Aqua) | q.s. |
| Butylene Glycol | 5 |
| Hydrolyzed Vegetable Protein | 1.23 |
| Trehalose | 0.69 |
| Glycerin | 0.69 |
| Tranexamic Acid | 5 |
| Niacinamide | 5 |
| 1,2-Hexandiol | 0.5 |
| Ethylhexylglycerin | 0.1 |
| Phenoxyethanol | 0.9 |
| Glycyrrhiza Glabra (Licorice) Root | 0.01 |
| Extract | |
| Polyacrylate-13 | 0.75 to 1.05 |
| Polyisobutene | 0.33 to 0.48 |
| Polysorbate 20 | 0.030 to 0.105 |
| Sorbitan isooctadecanoate | 0.045 to 0.075 |
| Dimethicone | 3.83 |
| Butylene Glycol | 0.30 |
| Decyl Glucoside | 0.01 |
| Caprylyl Glycol | 0.003 |
| Phenoxyethanol | 0.003 |
| Hexylene Glycol | 0.003 |
| Polysilicone-11 | 0.50 |
| Helianthus Annuus (Sunflower) Seed Oil, | 0.54 |
| Carthamus Tinctorius (Safflower) Seed | 0.16 |
| Oil | |
| Morus Alba (Mulberry) Leaf Extract | 0.16 |
| Squalane | 0.14 |
| Coconut Alkanes | 1.88 |
| Coco-Caprylate/Caprate | 0.12 |
| Kojic Acid Dipalmitate | 1.0 |
| Tetrahydrodiferuloylmethane | 0.25 |
| Hexyl Resorcinol | 0.4 |
| Alcohol Denat | 3 |
| Polymethylsilsesquioxane | 1.0 |
| Diamond Powder | 0.001 |
| Polysorbate 20 | 0.31 |
| Citric Acid | 0.222 |

### Example 5

Table 5 is an exemplary scrum formulation.

TABLE 5

| Component | Wt, % in formula |
|---|---|
| Water (Aqua) | q.s. |
| Butylene Glycol | 5 |
| Hydrolyzed Vegetable Protein | 1.231 |
| Trehalose | 0.692 |
| Glycerin | 0.692 |
| Tranexamic Acid | 5 |
| Niacinamide | 5 |
| 1,2-Hexandiol | 0.5 |
| Ethylhexylglycerin | 0.1 |
| Phenoxyethanol | 0.9 |
| Glycyrrhiza Glabra (Licorice) Root | 0.01 |
| Extract | |
| Polyacrylate-13 | 0.750 to 1.050 |
| Polyisobutene | 0.330 to 0.480 |
| Polysorbate 20 | 0.030 to 0.105 |
| Sorbitan isooctadecanoate | 0.045 to 0.075 |
| Dimethicone | 3.83 |
| Butylene Glycol | 0.30 |
| Decyl Glucoside | 0.010 |
| Caprylyl Glycol | 0.003 |
| Phenoxyethanol | 0.003 |
| Hexylene Glycol | 0.003 |
| Polysilicone-11 | 0.50 |
| Helianthus Annuus (Sunflower) Seed Oil, | 0.54 |
| Carthamus Tinctorius (Safflower) Seed | 0.16 |
| Oil | |
| Morus Alba (Mulberry) Leaf Extract | 0.16 |
| Squalane | 0.14 |
| Coconut Alkanes | 1.88 |
| Coco-Caprylate/Caprate | 0.12 |
| Kojic Acid Dipalmitate | 1 |
| Tetrahydrodiferuloylmethane | 0.25 |
| Hexyl Resorcinol | 0.4 |
| Alcohol Denat | 3 |
| Polysorbate 20 | 0.31 |
| Citric Acid | 0.22 |

### Example 6

Table 6 is an exemplary scrum formulation.

TABLE 6

| Component | Wt. % in formula |
|---|---|
| Water | q.s. |
| Cetyl Tranexamate Mesylate | 3.0 |
| Glycerin | 2.0 |
| Tamarindus Indica (Tamarind) Seed | 1.0 |
| Gum | |
| Caprylyl Glyceryl Ether (and) Glycerin | 0.5 |
| Caprylic/Capric Triglyceride | 5.0 |
| Cetearyl Alcohol | 2.0 |
| Glyceryl Stearate | 1.0 |
| Kojic Dipalmitate | 1.0 |
| Helianthus Annuus (Sunflower) Seed Oil | 0.54 |
| Carthamus Tinctorius (Safflower) Seed | 0.16 |
| Oil | |
| Morus Alba Leaf Extract | 0.16 |
| Squalane | 0.14 |
| Niacinamide | 6.35 |
| Tetrahydrodiferuloylmethane | 0.25 |
| Hexylresorcinol | 0.4 |
| Glycyrrhiza Glabra (Licorice) Root | 0.01 |
| Extract | |
| Propanediol | 4.0 |
| Trehalose (and) Hydrolyzed Vegetable | 3.0 |
| Protein | |
| Citric Acid (or) Sodium Hydroxide | q.s. to pH ~4.30-4.50 |

### Dark Spot Serum Clinical Study Synopsis

The advantageous and clinical efficacy of the formulation is demonstrated by using the following protocol:
Base size: 60 female subjects complete this study, 30 finish in one treatment cell, 30 finish the second treatment cell.
Washout: Subjects have discontinued antiaging products on the face for 2 weeks prior to study start subject to the exclusion criteria requirements.
Test products: RoC Derm Correxion Dark Spot Serum (Formula 6 of the present invention), comparison treatment (TBD)
Study duration: 16 weeks

### Inclusion/Exclusion

### Inclusion

Women between the ages of 35 and 65 years (inclusive) in general good health, who can read, understand, and sign the Informed Consent form and complete a brief medical history form.
1. All Fitzpatrick skin types with at least 3% of Fitzpatrick V and VI.
2. Individuals who indicate their willingness to participate in the study, follow directions, and stay on the study for the full 16 weeks.
3. Mild to moderate overall photodamage of the face with overall severity score of 3 to 6 on a 10-point scale with mild to moderate mottled hyperpigmentation (severity score of 3 to 6 on a 10-point scale).
4. Willing to avoid extended periods of sun exposure (especially from 11 AM to 4 PM). Willing to use an innocuous sunscreen if not able to avoid sun exposure
5. Willing to not have any cosmetics on the face during every study visit. If requested by the staff due to presence of makeup, willingness to wash in the clinic and undergo a 30-minute waiting period prior to having the study evaluations performed.
6. Willing to not introduce any new products (cleanser, cosmetics, etc.) into skincare regimen during the study and willing to not change any existing products during the study.
7. Individuals who are regular users of skin treatments.
8. Willing to sign a photography release.

### Exclusion

1. Any woman who do not meet the inclusion criteria.
2. Women who are pregnant, planning a pregnancy, or nursing a child.
3. Women with a known sensitivity to face/eye products
4. Women using systemic or topical anti-inflammatory agents with the exception of OTC acetaminophen (e.g., Tylenol), ibuprofen (e.g., Advil), or aspirin for 5 days prior to the start of testing.
5. Women who can not agree to not use systemic or topical anti-inflammatory agents, with the exception of OTC acetaminophen (e.g., Tylenol), ibuprofen (e.g., Advil), or aspirin during the course of the study.
6. Individuals using oral retinoids within 1 year prior to enrollment (e.g., Accutane®)
7. Individuals using topical prescription retinoids within 6 months prior to enrollment (e.g., Retin-A®, Avita®, Tazorac®, Avage®, Differin®)

| | First Visit | Week | | | | |
|---|---|---|---|---|---|---|
| Assessment | Baseline | 1 | 4 | 8 | 12 | 16 |
| Expert Grading of face for the following: mottled | [symbol] | [symbol] | [symbol] | [symbol] | [symbol] | [symbol] |
| hyperpigmentation, discrete pigmentation, undereye | | | | | | |
| fine lines, crow's feet, radiance, texture, uneven tone | | | | | | |
| VisiaCR with RBX imaging technology or equivalent | [symbol] | [symbol] | [symbol] | [symbol] | [symbol] | [symbol] |
| imaging and analysis: mottled hyperpigmentation, | | | | | | |
| dark spots, lines and wrinkles | | | | | | |
| Chromameter, such as DSM-4 Colorimeter or similar | [symbol] | [symbol] | [symbol] | [symbol] | [symbol] | [symbol] |
| to measure a target dark spot plus surrounding skin as | | | | | | |
| comparison | | | | | | |
| Subject questionnaire | [symbol] | [symbol] | [symbol] | [symbol] | [symbol] | [symbol] |
| Tolerability | [symbol] | [symbol] | [symbol] | [symbol] | [symbol] | [symbol] |
| Kinetic Moisturization (all 30 subjects): | | | | | | |
| Volar forearm/dry leg test sites; measurements with | | | | | | |
| TEWL, and corneometer at baseline, 15 mins after | | | | | | |
| product application, 4, 8, and 24 hrs | | | | | | |

* Includes baseline assessment and applying test product if not excluded.

### Self-Assessment Questionnaire (at Baseline):

After subjects apply test product at the clinic after baseline procedures have been completed (5 pt. scale, Agree strongly, agree, neither agree nor disagree, disagree, disagree strongly)
1. My skin is smoother
2. Reduces the appearance of fine lines
3. Brightens my skin
4. My skin tone is more even
5. Dark spots are reduced
6. Skin imperfections are improved
7. My skin is hydrated
8. Leaves my skin softer
9. Skin looks healthier
10. This product absorbs quickly into skin
11. This product has an appealing texture
12. Product is gentle

### Self Assessment Questionnaire (Day 5, Weeks 4 and 8):

Questions regarding the appearance of facial skin (5 pt. scale, Agree strongly, agree, neither agree nor disagree, disagree, disagree strongly):
1. My skin looks more radiant/brighter
2. My skin is healthy looking
3. My skin tone looks more even
4. My skin is softer
5. My skin is smoother
6. My skin tone is more even
7. Dark spots are reduced
8. Skin imperfections are improved
9. My skin feels hydrated
10. Reduces the appearance of fine lines
11. Reduces wrinkles
12. This product absorbs quickly into skin
13. This product has an appealing texture
14. Product is gentle

Open ended questions-like, dislike, would you recommend . . .

### Quality of Life Questions (Week 12 Only)

1. I can delay a visit to the dermatologist
2. Delivers results better than expected with skincare
3. I would use this instead of a prescription skincare product
4. I can use less makeup after using this product
5. I feel more confident about the appearance of my skin
6. Using this product makes me optimistic about my appearance
Open ended questions-like, dislike, would you recommend . . .

Further aspects of the present disclosure are provided by the subject matter of the following clauses.

A composition for evening skin tone that includes at least one tyrosine formation inhibitor, at least one tyrosinase inhibitor, and at least one agent that reduces melanosome transfer from melanocytes to keratinocytes, wherein the composition does not comprise hydroquinone.

The composition of the preceding clause, wherein the composition includes the at least one tyrosine formation inhibitor, the at least one tyrosinase inhibitor, and the at least one agent that reduces melanosome transfer from melanocytes to keratinocytes in an amount sufficient to reduce the number, size, and/or intensity of dark spots.

The composition of any preceding clause, wherein: the at least one tyrosine formation inhibitor comprises kojic acid or an ester thereof, the at least one tyrosinase inhibitor comprises tranexamic acid, an ester of tranexamic acid, licorice root extract, hexyl resorcinol, or a combination thereof, and the at least one agent that reduces melanosome transfer from melanocytes to keratinocytes comprises niacinamide.

The composition of any preceding clause, wherein the at least one tyrosine formation inhibitor includes kojic acid or an ester thereof.

The composition of any preceding clause, wherein the at least one tyrosinase inhibitor is tranexamic acid, an ester of tranexamic acid, licorice root extract, hexyl resorcinol, or a combination thereof.

The composition of any preceding clause, wherein the at least one tyrosinase inhibitor is a combination of tranexamic acid, an ester of tranexamic acid, licorice root extract, hexyl resorcinol, or a combination thereof.

The composition of any preceding clause, wherein the at least one tyrosinase inhibitor is cetyl tranexamate mesylate, licorice root extract, hexyl resorcinol, or a combination thereof.

The composition of any preceding clause, wherein the at least one tyrosinase inhibitor is a combination of cetyl tranexamate mesylate, licorice root extract, hexyl resorcinol.

The composition of any preceding clause, wherein: the at least one agent that reduces melanosome transfer from melanocytes to keratinocytes is niacinamide, malassezin, or a and combination thereof.

The composition of any preceding clause, wherein: the at least one agent that reduces melanosome transfer from melanocytes to keratinocytes is niacinamide.

The composition of any preceding clause, wherein the composition includes kojic acid or an ester of kojic acid, tranexamic acid or an ester of tranexamic acid, licorice root extract, hexyl resorcinol, and niacinamide.

The composition of any preceding clause, wherein the kojic acid or ester of kojic acid is kojic dipalmitate.

The composition of any preceding clause, wherein the tranexamic acid or ester of tranexamic acid is cetyl tranexamate mesylate.

The composition of any preceding clause, wherein the tranexamic acid or ester of tranexamic acid is tranexamic acid.

The composition of any preceding clause, wherein the composition includes: from 0.2 wt. % to 2.0 wt. % kojic dipalmitate, from 1.0 wt. % to 10.0 wt. % tranexamic acid or an ester of tranexamic acid, from 0.001 wt. % to 0.05 wt. % licorice root extract, from 0.10 wt. % to 1.0 wt. % hexyl resorcinol, and from 1.0 wt. % to 15.0 wt. % niacinamide.

The composition of any preceding clause, wherein the composition includes about 1.0 wt. % kojic dipalmitate, about 5.0 wt. % tranexamic acid, about 0.01 wt. % licorice root extract, about 0.40 wt. % hexyl resorcinol, and about 5.0 wt. % niacinamide.

The composition of any preceding clause, wherein the composition includes about 1.0 wt. % kojic dipalmitate, about 3.0 wt. % cetyl tranexamate mesylate, about 0.01 wt. % licorice root extract, about 0.40 wt. % hexyl resorcinol, and about 6.35 wt. % niacinamide.

The composition of any preceding clause, wherein the composition does not contain mandelic acid or an ester thereof.

The composition of any preceding clause, wherein the composition further includes at least one exfoliating enzyme.

The composition of any preceding clause, wherein the composition does not contain azelaic acid or an ester thereof.

The composition of any, wherein the composition further includes citric acid.

The composition according to any preceding clause, wherein the composition includes a retinoid such as retinol.

The composition of any preceding clause, wherein the composition does not contain retinol.

The composition according to any preceding clause, wherein the composition also includes one or more exfoliants.

The composition according to the preceding clause, wherein the exfoliants are chosen from chemical exfoliants, enzyme exfoliants, and combinations thereof.

The composition according to the preceding clause, wherein the chemical exfoliants are chosen from mandelic acid, glycolic acid, lactic acid, malic acid, tartaric acid, citric acid, salicylic acid, lactobionic acid, gluconolactone, galactose, and combinations thereof.

The composition according to any preceding clause, further including an enzyme exfoliants are chosen from a trypsin, a collagenase, and combinations thereof.

The composition of any preceding clause, further including one or more excipients selected from emulsifiers, surfactants, rheology modifiers, humectants, emollients, skin conditioning agents, pH modifiers and buffers, preservatives, antimicrobial agents, fragrance, antioxidants, antiaging agents, anti-wrinkle agents, film-forming agents.

Use of the composition of any preceding clause in the manufacture of a cosmetic for evening skin tone.

Use according to the preceding clause wherein the composition is applied to the skin in an amount sufficient to reduce the number, size, or intensity of dark spots.

A method for persistently evening skin tone comprising applying the composition according to any preceding clause.

The method of the preceding clause, wherein the composition is applied to the skin in an amount sufficient to reduce the number, size, or intensity of dark spots.

The method of the preceding clause, wherein the reduction of dark spots is at least as effective as treatment with a hydroquinone-containing composition.

The method of any preceding clause, wherein the reduction of dark spots or intensity of dark spots is at least as effective as treatment with a hydroquinone-containing composition that does not contain one or more of kojic acid or an ester of kojic acid, tranexamic acid or an ester of tranexamic acid, licorice root extract, hexyl resorcinol, and niacinamide.

The method of any preceding clause, wherein the reduction of dark spots or intensity of dark spots is at least as effective as treatment with a hydroquinone-containing composition that does not contain two or more of kojic acid or an ester of kojic acid, tranexamic acid or an ester of tranexamic acid, licorice root extract, hexyl resorcinol, and niacinamide.

The method of any preceding clause, wherein the reduction of dark spots or intensity of dark spots is at least as effective as treatment with a hydroquinone-containing composition that does not contain kojic acid or an ester of kojic acid, tranexamic acid or an ester of tranexamic acid, licorice root extract, hexyl resorcinol, and niacinamide.\

The method of any preceding clause, wherein the reduction of dark spots is more effective than treatment with a hydroquinone-containing composition.

The method of any preceding clause, wherein the reduction of dark spots or intensity of dark spots is more effective than treatment with a hydroquinone-containing composition that does not contain one or more of kojic acid or an ester of kojic acid, tranexamic acid or an ester of tranexamic acid, licorice root extract, hexyl resorcinol, and niacinamide.

The method of any preceding clause, wherein the reduction of dark spots or intensity of dark spots is more effective than treatment with a hydroquinone-containing composition that does not contain two or more of kojic acid or an ester of kojic acid, tranexamic acid or an ester of tranexamic acid, licorice root extract, hexyl resorcinol, and niacinamide.

The method of any preceding clause, wherein the reduction of dark spots or intensity of dark spots is more effective than treatment with a hydroquinone-containing composition that does not contain kojic acid or an ester of kojic acid, tranexamic acid or an ester of tranexamic acid, licorice root extract, hexyl resorcinol, and niacinamide.

The method according to any preceding clause, wherein the composition is applied at a frequency and duration sufficient for an even skin tone to persistent for at least 30 minutes, at least 1 hour, at least 2 hours, at least 4 hours, at least 12 hours, or at least 1 day after removing the composition from the skin.

The method according to any preceding clause, wherein the composition is applied at a frequency and duration sufficient for an even skin tone to persistent for at least 1, at least 2 days, at least 1 week, at least 2 weeks, or at least one month after application of the composition is ceased.

The method according to any preceding clause, wherein the composition is applied to the skin for once or twice a day.

The method according to any preceding clause, wherein the composition is applied to the skin each day for at least 1 day, at least 2 days, at least 1 week, at least 2 weeks, or at least 1 month.

The embodiments illustrated and discussed in this disclosure are intended only to teach those skilled in the art the best way known to the inventors to make and use the invention. Nothing in this disclosure should be considered as limiting the scope of the present invention. All examples presented are representative and non-limiting. The above-described embodiments of the disclosure may be modified or varied, without departing from the disclosure, as appreciated by those skilled in the art in light of the above teachings. It is therefore to be understood that, within the scope of any claims supported by the disclosure and their equivalents, the invention may be practiced otherwise than as specifically described.

What is claimed is:

1. A composition for evening skin tone comprising:
at least one tyrosine formation inhibitor,
at least one tyrosinase inhibitor, and
at least one agent that reduces melanosome transfer from melanocytes to keratinocytes, wherein the composition does not comprise hydroquinone.

2. The composition of claim 1, wherein the composition comprises the at least one tyrosine formation inhibitor, the at least one tyrosinase inhibitor, and the at least one agent that reduces melanosome transfer from melanocytes to keratinocytes in an amount sufficient to reduce number, size, and/or intensity of dark spots.

3. The composition of claim 1, wherein:
the at least one tyrosine formation inhibitor comprises kojic acid or an ester thereof,
the at least one tyrosinase inhibitor comprises tranexamic acid, an ester of tranexamic acid, licorice root extract, hexyl resorcinol, or a combination thereof, and
the at least one agent that reduces melanosome transfer from melanocytes to keratinocytes comprises niacinamide.

4. The composition of claim 1, wherein the at least one tyrosine formation inhibitor comprises kojic dipalmitate.

5. The composition of claim 1, wherein the at least one tyrosinase inhibitor comprises tranexamic acid or cetyl tranexamate mesylate, licorice root extract, hexyl resorcinol, or a combination thereof.

6. The composition of claim 1, wherein the at least one tyrosinase inhibitor comprises a combination of tranexamic acid or cetyl tranexamate mesylate, licorice root extract, and hexyl resorcinol.

7. The composition of claim 1, wherein the at least one agent that reduces melanosome transfer from melanocytes to keratinocytes comprises niacinamide.

8. The composition of claim 1, wherein the composition comprises kojic acid or an ester of kojic acid, tranexamic acid or an ester of tranexamic acid, licorice root extract, hexyl resorcinol, and niacinamide.

9. The composition of claim 1, wherein the composition comprises kojic dipalmitate, tranexamic acid or cetyl tranexamate mesylate, licorice root extract, hexyl resorcinol, and niacinamide.

10. The composition of claim 1, wherein the at least one tyrosine formation inhibitor comprises kojic acid or an ester thereof.

11. The composition of claim 1, wherein the composition comprises:
from 0.2 wt. % to 2.0 wt. % kojic dipalmitate,
from 1.0 wt. % to 10.0 wt. % tranexamic acid or an ester of tranexamic acid,
from 0.001 wt. % to 0.05 wt. % licorice root extract,
from 0.10 wt. % to 1.0 wt. % hexyl resorcinol, and
from 2.5 wt. % to 7.5 wt. % niacinamide.

12. The composition of claim 11, wherein the composition comprises about 1.0 wt. % kojic dipalmitate, about 5.0 wt. % tranexamic acid, about 0.01 wt. % licorice root extract, about 0.40 wt. % hexyl resorcinol, and about 5.0 wt. % niacinamide.

13. The composition of claim 11, wherein the composition comprises about 1.0 wt. % kojic dipalmitate, about 3.0 wt. % cetyl tranexamate mesylate, about 0.01 wt. % licorice root extract, about 0.40 wt. % hexyl resorcinol, and about 6.35 wt. % niacinamide.

14. A method for evening skin tone comprising applying the composition of claim 1 to the skin.

15. The method of claim 14, wherein the composition is applied to the skin in an amount sufficient to reduce number, size, and/or intensity of dark spots.

16. The method of claim 15, wherein the reduction of dark spots is at least as effective as treatment with a hydroquinone-containing composition.

17. The method of claim 14, wherein the composition is applied at a frequency and duration sufficient for an even skin tone to persistent for at least 30 minutes, at least 1 hour, at least 2 hours, at least 4 hours, at least 12 hours, or at least 1 day after removing the composition from the skin.

18. The method of claim 14, wherein the composition is applied at a frequency and duration sufficient for an even skin tone to persistent for at least 1, at least 2 days, at least 1 week, at least 2 weeks, or at least one month after application of the composition is ceased.

19. The method of claim 14, wherein the composition is applied to the skin for once or twice a day.

20. The method of claim 14, wherein the composition is applied to the skin each day for at least 1 day, at least 2 days, at least 1 week, at least 2 weeks, or at least 1 month.
